SEQUENCE LISTING (11 sequences)

SEQ ID NO: 1 - protein, 10 aa, Homo sapiens
EQKLISEEDL

SEQ ID NO: 2 - protein, 14 aa, Simian parainfluenza virus 5
GKPIPNPLLGLDST

SEQ ID NO: 3 - DNA, 20 bp, Artificial
  Oligonucleotide primer
cgccaagaacctcatcatct

SEQ ID NO: 4 - DNA, 20 bp, Artificial
  Oligonucleotide primer
cgtcaatgtccatgttggag

SEQ ID NO: 5 - DNA, 42 bp, Artificial
  Oligonucleotide primer
ctgcaggagcagaaactcatctctgaagaggatctgctgcag

SEQ ID NO: 6 - DNA, 19 bp, Artificial
  Oligonucleotide primer
atggtcctgtgtgtgtcac

SEQ ID NO: 7 - DNA, 19 bp, Artificial
  Oligonucleotide primer
caggggaactttattgaga

SEQ ID NO: 8 - DNA, 71 bp, Artificial
  Oligonucleotide primer
tggtcccaggcagcagttagcccgccgcccgcctgtgtgtccccagagccatggagatgttccaggggctg

SEQ ID NO: 9 - DNA, 71 bp, Artificial
  Oligonucleotide primer
tagcgttcggcctgctctgccagcttggccttctggatcagactggctctttaccaatgcttaatcagtga

SEQ ID NO: 10 - DNA, 73 bp, Artificial
  Oligonucleotide primer
tgcagtgagccgagatctcgccactgcactactccagcctgggcgacagagcttacgccccgccctgccactc

SEQ ID NO: 11 - DNA, 70 bp, Artificial
  Oligonucleotide primer
ggatatgggagccagccacattcatacagggcacacatgaacacacacatgtcaaacatgagaattacaa

(19) United States
(12) Patent Application Publication
(10) Pub. No.: US 2007/0217999 A1
(43) Pub. Date: Sep. 20, 2007

(54) Excretable Reporter Systems

(75) Inventors: Anthony John Clark, Midlothian (GB); Helen Elizabeth Clark, Midlothian (GB); Charles Roland Wolf, Dundee (GB); Christopher Bruce Alexander Whitelaw, Midlothian (GB); Kenneth Brown, Dundee (GB); Simon Martin Temperley, Dundee (GB)

Correspondence Address: MYERS BIGEL SIBLEY & SAJOVEC, PO BOX 37428, RALEIGH, NC, 27627, US

(73) Assignee: Roslin Institute (Edinburgh), Midlothian, Scotland (GB)

(21) Appl. No.: 10/572,975

(22) PCT Filed: Sep. 23, 2004
(86) PCT No.: PCT/GB04/04054

§ 371 Date: May 10, 2007

(30) Foreign Application Priority Data
Sep. 23, 2003 (GB) 0322196.7

(51) Int. Cl. C12N 15/00; A61K 49/00; C12N 5/06; C12Q 1/68

(52) U.S. Cl. 424/9.1; 435/320.1; 435/325; 435/6; 435/69.1; 800/13; 800/14; 800/18; 800/4

(57) ABSTRACT

The present invention provides a reporter system comprising a reporter gene encoding a reporter protein that is secretable from cells in which it produced or expressed either in vitro or in vivo and excretable in a body fluid from whole animals comprising such systems. The reporter system is useful for the detection of gene activation events or biochemical changes related to, or that occur, as a result of altered metabolic or disease status or toxicological stress in toxicological screening.

The present invention relates to reporter systems comprising a reporter gene encoding a reporter protein that is secretable from cells in which it produced or expressed either in vitro or in vivo and excretable from whole animals comprising such systems. The reporter system is for the detection of gene activation events or biochemical changes related to, or that occur, as a result of altered metabolic or disease status or toxicological stress both for use, particularly but not exclusively, in toxicological screening. The present invention also provides methods of making such systems and uses thereof.

### BACKGROUND TO THE INVENTION

Genes encode proteins. It is estimated that there are at least 3×10⁴ genes in the vertebrate genome but for a given cell only a subset of the total number of genes is active, with the subset differing between cells of different types and between different stages of development and differentiation (Cho & Campbell Trends Genet. 16 409-415 (2000); Velculescu et al Trends Genet. 16 423-425 (2000)). The DNA regulatory elements associated with each gene governs the decision as to which genes are active and which are not. Although comprising a number of defined elements these DNA sequences are collectively termed promoters (Tjian & Maniatis Cell 77 5-8 (1994); Bonifer, Trends Genet. 16 310-315 (2000); Martin, Trends Genet 17 444-448 (2001)).

Gene activation occurs primarily at the transcriptional level. Transcriptional activity of a gene may be measured by a variety of approaches including RNA polymerase activity, mRNA abundance or protein production (Takano et al., 2002). These approaches are limited in that they require development of an assay suitable to each individual mRNA or protein product. To facilitate comparison of different promoters, rather than assaying individual gene products, reporter genes are often used (Sun et al Gene Ther. 8 1 572-1579 (2001); Franco et al Eur. J. Morphol. 39 169-191 (2001); Hadjantonakis & Nagy, Histochem. Cell. Biol. 115 49-58 (2001); Gorman Mol. Cell. Biol. 2 1044-1051 (1982); Barash and Reichenstein, 2002; Zhang et al., 2001.)

The product (mRNA or protein) of a reporter gene allows an assessment of the transcriptional activity of a particular gene and can be used to distinguish cells, tissues or organisms in which the event has occurred from those in which it has not. On the whole, reporter genes are foreign to the host cell or organism, allowing their activity to be easily distinguished from the activity of endogenous genes. Alternatively the reporter may be marked or tagged so as to make it distinct from host genes.

Reporter genes are linked to the test promoter, enabling activity of the promoter gene to be determined by detecting the presence of the reporter gene product. Therefore, the main prerequisite for a reporter gene product is that it is easy to detect and quantify. In some cases, but not all, the reporter gene has enzymatic activity that catalyses the conversion of a substrate into a measurable product.

A classical example is the bacterial chloramphenicol acetyl transferase (CAT) gene. CAT activity can be measured in cell extracts as conversion of added non-acetylated chloramphenicol to the acetylated form of chloramphenicol by chromatography (Gorman Mol. Cell. Biol. 2 1044-1051 (1982)). Further examples of enzymatic reporters include alkaline phosphatase, β-galactosidase, thymidine kinase, neomycin resistance and growth hormone. Similar strategies enable the use of the firefly luciferase gene as a reporter. However, in this instance it is the light produced by bioluminescence of the luciferin substrate that is measured.

Some reporters also benefit from the visual detection assays that allow in situ analysis of reporter activity. A frequently used example would be β-galactosidase (Lac Z), where the addition of an artificial substrate, X-gal, enables reporter activity to be detected by the appearance of blue coloration in the sample. As it is accumulative it effectively provides an historical record of its induction. This is particularly useful for measuring transient responses where a promoter is activated for only a short time before being rapidly inactivated. This reporter has been successfully used both in cultured cells and in vivo (Campbell et al J. Cell. Biol. 109 2619-2625 (1996)), though its suitability for in vivo use has been questioned in some reports (Chevalier-Mariette et al., Genome Biol. 4 R53 (2003); Sanchez-Ramnos et al Cell Transplant. 9 657-667 (2000); Montoliu et al Transgenic Res. 9 237-239 (2000); Cohen-Tannoudji et al Transgenic Res. 9 233-235 (2000)). It has been demonstrated that Lac Z in combination with fluorescent substrates can enable the sorting of cells that express the reporter by use of a fluorescence-activated cell sorter (FACS) (Fiering et al Cytometry 12 291-301 (1991)).

In other systems, the reporter product itself is directly detected, removing the need for a substrate. Green fluorescent protein has become one of the most commonly used examples of this category of reporter (Ikawa et al Curr. Top. Dev. Biol. 44 1-20 (1997)). This autofluorescing protein was derived from the bioluminescent jellyfish Aequoria Victoria. Several colour spectral variants of this reporter have been developed (Hadjantonakis & Nagy, Histochem. Cell. Biol. 115 49-58 (2001)).

Recently reporter systems based on energy emission systems have been developed. These include single photon emission computed tomography (SPECT) and positron emission tomography (PET) though these require the introduction of a radiolabelled isotope probe in to the host cell or animal that is then modified by the target reporter gene. For example the PET system measures reporter sequestering of the positron-emitting probe (Sun et al Gene Ther. 8 1572-1579 (2001)).

Many tried and tested reporter systems have been developed but nevertheless they share certain limitations. Those based on prokaryote genes often suffer poor expression in transgenic mammals (Montoliu et al Transgenic Res. 9 237-238 (2000); Cohen-Tannoudji et al Transgenic Res. 9 233-235 (2000)). Furthermore the presence of prokaryote DNA sequences has been implicated in the suppression of expression from adjacent eukaryote transgenes as have the presence of intronless, cDNA based eukaryote gene sequences (Clark et al., 1997).

Most of the current reporters, whilst useful for monitoring expression under certain circumstances, have certain limitations. Many accumulate in cells and are not useful for monitoring changes in promoter activation over time. Perhaps more importantly detection of expression necessitates the fixing of cultured cells or the sacrifice of transgenic animals, thus limiting reporters to invasive detection strategies. There are a few exceptions; These include the use of growth hormone (Bchini et al Endocrinology 128 539-546 (1991)). However its high biological activity effectively limit its widespread applicability. GFP has been detected in whole animals and though possessing relatively low biological activity its use has so far been limited to neonatal and nude mice in which both internal tissue and dermal fluorescence are more readily observed. In addition there has been a report that GFP is cytotoxic (Liu et al Biochem. Biophys. Res. Comm. 260 712-717 (1999)). Although reporter systems based on tomography allow monitoring of reporter expression in internal tissues they require addition of exogenously added substrates that could potentially confound results by influencing expression of the reporter. Additionally they can lack the sensitivity required for quantitative analysis of reporter expression.

There is therefore a need for a reporter system that overcomes some or all of these limitations. In the first instance the reporter should be secreted (from the cell in which it is expressed or produced) and preferably excreted (from the whole animal) so that advantageously the system is non-invasive inasmuch as its detection does not involve addition of an external substrate or sacrifice of transgenic animals. In addition, the system should be biologically neutral with regard to the test expression system so that no phenotypic effects either confound readout from the system or affect the health of the transgenic animal.

A system satisfying such requirements has now been found. The non-invasive reporters of the present invention comprise characteristics which favour secretion from the cell where it is produced or expressed and excretion of the gene product or metabolite into a body fluid, relative stability and distinction from native molecules.

### STATEMENT OF THE INVENTION

In its broadest aspect the present invention provides a biological reporter system that permits non-invasive measurement of biochemical changes arising as result of toxic insult/stress, constitutive or induced disease states and/or altered metabolic status.

According to a first aspect of the invention, there is provided a nucleic acid construct comprising a nucleic acid sequence comprising a reporter gene encoding a reporter protein that is secretable as a protein or product from a cell where it is expressed or produced and that is excretable from a whole animal.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

Reference herein to protein or product is intended to include: protein complexes or fragments; enzymes; enzymatic products or conjugates; primary, secondary or further metabolites and/or salts thereof; non-biological products that are released by direct or secondary effects on the expression of the reporter gene product; hormones or; antibodies.

Throughout the present specification the reporter system proteins of the present invention are conveniently referred to as a secreted/excreted proteins or products. It will be appreciated by the skilled man that this term relates to the reporter protein or product being firstly secreted from a cell where it is either expressed or produced and subsequently being excreted into a body fluid from where it may be measured or monitored or assayed.

Preferably, the secreted/excreted protein/product may be produced as a result of modulated gene transcription.

Alternatively, the secreted/excreted reporter protein/product may be produced and secreted/excreted as a result of increased reporter translation for example as a result of increased stability or decreased turnover of m RNA.

As a yet further alternative, the reporter protein/product may be as the result of post translational modification such as increased reporter stability through removal of polyubiquination or alternatively the reporter protein/product may be as the result of accumulation or excretion of small molecule metabolites.

We have found surprisingly that the activity of SEAP, a secreted version of alkaline phosphatase, can be induced both in vitro and in vivo and that it is excreted in body fluids such as the blood and urine of transgenic animals. Accordingly, SEAP is an example of a secreted/excreted reporter protein of the present invention. However it will be apparent that other reporter proteins having similar characteristics described herein after will also be suitable for the present invention.

Therefore in one embodiment of the invention the nucleic acid construct comprises a nucleic acid sequence comprising a reporter gene encoding the SEAP reporter protein that is secretable as a protein or product from a cell where it is expressed or produced and that is excretable from a whole animal.

In a further embodiment of the invention the reporter protein or product may comprise a peptide tag such as an epitope tag or a tag which may posses enzymatic activity to convert a substrate to a form that is readily detectable by an assay. This embodiment of the invention advantageously provides for multiple reporter systems in a single cell or single animal.

It will be appreciated that the position of the peptide tag may be at the amino terminal or carboxy terminal or inserted internally with respect to the amino acid sequence of the reporter, and that in the instance of the tag being an epitope tag that it is recognised by its cognate antibody irrespective of its location in the reporter protein.

The epitope tag may be a defined amino acid sequence from a protein with a fully characterised cognate antibody. The skilled person can select such epitopes based on sequences identified as possessing antigenic properties. In certain embodiments of the invention the epitope tag may be the amino acid sequence below from the c-myc oncogene (Evans et al Mol. Cell. Biol. 5 3610-3616 (1985)):

(SEQ ID NO:1)

-Glu-Gln-Lys-Leu-Ile-Ser-Glu-Glu-Asp-Leu-

or it may be the amino acid sequence from the simian virus V5 protein (Southern et al J. Gen. Virol. 72 1551-1557 (1991)), shown below:

(SEQ ID NO:2)

-Gly-Lys-Pro-Ile-Pro-Asn-Pro-Leu-Leu-Gly-Leu-Asp-
Ser-Thr-

In certain embodiments of the invention, the epitope may be selected from but not limited to the c-myc and V5 proteins.

In some instances the peptide tag may possess enzymatic activity that converts a substrate to a form that is readily detectable by an assay, the tag may have kinase activity specifying phosphorylation of another protein or peptide substrate that could be added to the secreted or excreted analyte along with a phosphate group donor. Detection could be achieved using an immunological assay based on detection by an antibody specifically recognising the phosphorylated version of the tagged reporter protein. Such assays include ELISA, Western blot, RIA and fluorescence polarisation. Alternatively the use a released labelled product for example, phosphate radiolabelled with an isotope of phosphorous such as ³²P or ³³P, which could be measured by fluorometric, radioactive or calorimetric means. Other enzymic modifications include for example acetylation, sulphation and glycosylation.

Other embodiments of this aspect could include, for example site of interaction with protein other than antibody e.g. lectin binding site, or modification of tag by e.g. addition of amino acid multimer such as polylysine; or incorporation of a fluorochrome.

According to the various embodiments of this aspect of the invention, the reporter gene may be associated with a promoter. The promoter will preferably be of mammalian origin, but also may be from a non-mammalian animal, plant, yeast or bacteria. The promoter may be selected from but is not limited to promoter elements of the following inducible genes:
whose expression is modified in response to disturbances in the homeostatic state of DNA in the cell. These disturbances may include chemical alteration of nucleic acids or precursor nucleotides, inhibition of DNA synthesis and inhibition of DNA replication or damage to DNA. The sequence can be selected from but not limited to the group consisting of c-myc (Hoffman et al Oncogene 21 3414-3421), p21/WAF-1 (El-Diery Curr. Top. Microbiol. Immunol. 227 121-137 (1998); El-Diery Cell Death Differ. 8 1066-1075 (2001); Dotto Biochim. Biophys. Acta 1471 43-56 (2000)), MDM2 (Alarcon-Vargas & Ronai Carcinogenesis 23 541-547 (2002); Deb & Front Bioscience 7 235-243 (2002)), Gadd45 (Sheikh et al Biochem. Pharmacol. 59 43-45 (2000)), FasL (Wajant Science 296 1635-1636 (2002)), GAHSP40 (Hamajima et al J. Cell. Biol. 84 401-407 (2002)), TRAIL R2/DR5 (Wu et al Adv. Exp. Med. Biol. 465 143-151 (2000); El-Diery Cell Death Differ. 8 1066-1075 (2001)), BTG2/PC3 (Tirone et al J. Cell. Physiol. 187 155-165 (2001));
whose transcription is modified in response to oxidative stress or hypoxia. The sequence can be selected from but not limited to the group consisting of MNSOD and/or CuZnSOD (Halliwell Free Radic. Res. 31 261-272 (1999); Gutteridge & Halliwell Ann. NY Acad. Sci. 899 136-147 (2000)), I□B (Ghosh & Karin Cell 109 Suppl., S81-96 (2002)), ATF4 (Hai & Hartman Gene 273 1-11 (2001)), xanthine oxidase (Pristos Chem. Biol. Interact. 129 195-208 (2000)), COX2 (Hinz & Brune J. Pharmacol. Exp. Ther. 300 376-375 (2002)), iNOS (Alderton et al Biochem. J. 357 593-615 (2001)), Ets-2 (Bartel et al Oncogene 19 6443-6454 (2000)), FasL/CD95L (Wajant Science 296 1635-1636 (2002)), □GCS (Lu Curr. Top. Cell. Regul. 36 95-116 (2000); Soltaninassab et al J. Cell. Physiol. 182 163-170 (2000)), ORP150 (Ozawa et al Cancer Res. 61 4206-4213 (2001); Ozawa et al J. Biol. Chem. 274 6397-6404 (1999)).
whose expression is modified in response to hepatotoxic stress. The sequence can be selected from but not limited to the group consisting of Lrg-21 (Drysdale et al Mol. Immunol. 33 989-998 (1996)), SOCS-2 and/or SOCS-3 (Tollet-Egnell et al Endocrinol. 140 3693-3704 (1999), PAI-1 (Fink et al Cell. Physiol. Biochem. 11 105-114 (2001)), GBP28/adiponectin (Yoda-Murakami et al Biochem. Biophys. Res. Commun. 285 372-377 (2001)), □-1 acid glycoprotein (Komori et al Biochem Pharmacol. 62 1391-1397 (2001)), metallothioneine I (Pamiter et al Mol. Cell. Biol. 13 5266-5275 (1993)), metallothioneine II (Schlager & Hart App. Toxicol. 20 395-405 (2000)), ATF3 (Hai & Hartman Gene 273 1-11 (2001)), IGFbp-3 (Popovici et al J. Clin. Endocrinol. Metab. 86 2653-2639 (2001)), VDGF (Ido et al Cancer Res. 61 3016-3021 (2001)) and HIF1 [symbol] (Tacchini et al Biochem. Pharmacol. 63 139-148 (2002)).
whose expression is modified in response to a pro-apoptotic stimulus. The sequence can be selected from but not limited to the group consisting of Gadd 34 (Hollander et al J. Biol. Chem. 272 13731-13737 (1997)), GAHSP40 (Hamajima et al J. Cell. Biol. 84 401-407 (2002)), TRAIL-R2/DR5 (Wu et al Adv. Exp. Med. Biol. 465 143-151 (2000); El-Diery Cell Death Differ. 8 1066-1075 (2001)), c-fos (Teng Int. Rev. Cytol. 197 137-202 (2000)), CHOP/Gadd153 (Talukder et al Oncogene 21 4280-4300 (2002)), APAF-1 (Cecconi & Gruss Cell. Mol. Life Sci. 5 1688-1698 (2001)), Gadd45 (Sheikh et al Biochem. Pharmacol. 59 43-45 (20000, BTG2/PC3 (Tirone J. Cell. Physiol. 187 155-165 (2001)), Peg3/Pwl (Relaix et al Proc. Nat'l Acad. Sci. USA 97 2105-2110 (2000)), Siah la (Maeda et al FEBS Lett. 512 223-226 (2002)), S29 ribosomal protein (Khanna et al Biochem. Biophys. Res. Commun. 277 476-486 (2000)), FasL/CD95L (Wajant Science 296 1635-1636 (2002)), tissue tranglutaminase (Chen & Mehta Int. J. Cell. Biol. 31 817-836 (1999)), GRP78 (Rao et al FEBS Lett. 514 122-128 (2002)), Nur77/NGFI-B (Winoto Int. Arch. Allergy Immunol. 105 344-346 (1994)), CyclophilinD (Andreeva et al Int. J. Exp. Pathol. 80 305-315 (1999)), p 73 (Yang et al Trends Genet. 18 90-95 (2002)) and Bak (Lutz Biochem. Soc. Trans. 28 51-56 (2000)).
whose expression is modified in response to the administration of chemicals or drugs or other xenobiotic agents. The sequence can be selected from but not limited to the list comprised of xenobiotic metabolising cytochrome p450 enzymes from the 2A, 2B, 2C, 2D, 2E, 2S, 3A, 4A and 4B gene families (Smith et al Xenobiotica 28 1129-1165 (1998); Honkaski & Negishi J. Biochem. Mol. Toxicol. 12 3-9 (1998); Raucy et al J. Pharmacol. Exp. Ther. 302 475-482 (2002); Quattrochi & Guzelian Drug Metab. Dispos. 29 615-622 (2001)).
whose expression is modified in response to disease states either natural, modelled or induced. These diseases can be selected from but not limited to the list comprised of obesity, compromised immunity, degenerative neurological disorders, cancer, cardiovascular, inflammatory diseases, genetic diseases or metabolic disorders.

The promoter element may comprise a contiguous “wild-type” sequence or it may be a synthetic promoter sequence comprised of a minimal eukaryote consensus promoter operatively linked to one or more sequence elements known to confer transcriptional inducibility in response to specific stimulus. A minimal eukaryotic consensus promoter is one that will direct transcription by eukaryotic polymerases only if associated with functional promoter elements or transcription factor binding sites. An example of which is the PhCMV*-1 (Furth et al Proc. Nat'l Acad. Sci. USA 91 9302-9306 (1994)). Sequence elements known to confer transcriptional induction in response to specific stimulus include promoter elements (Montoliu et al Proc. Nat'l Acad. Sci. USA 92 4244-4248 (1995)) or transcription factor binding sites; these will be chosen from but are not limited to the list comprising the aryl hydrocarbon (Ah)/Ah nuclear translocator (ARNT) receptor response element, the antioxidant response element (ARE), the xenobiotic response element (XRE).

According to a further aspect of the invention there is provided a nucleic acid construct comprising a stress inducible promoter operatively isolated from a nucleic acid sequence encoding a reporter protein that is secretable from a cell where it is expressed as a protein or product and that is excreted from a whole animal, said sequence being flanked by nucleic acid sequences recognised by a site specific recombinase, or by insertion such that it is inverted with respect to the transcription unit encoding a secreted/excreted reporter protein. The recombinase recognition sites are arranged in such a way that the isolator sequence is deleted or the inverted promoter's orientation is reversed in the presence of the recombinase. The construct also comprises a nucleic acid sequence comprising a tissue specific promoter operatively linked to a gene encoding the coding sequence for the site specific recombinase.

This aspect allows for detecting reporter transgene induction in specified tissues only. By controlling the appropriate recombinase expression using a tissue specific promoter, the inducible transgene will only be viable in those tissues in which the promoter is active. For example, by driving recombinase activity from a liver specific promoter, only the liver will contain re-arranged reporter construct, and hence will be the only tissue in which reporter induction can occur.

The recombination event producing an active reporter transcription unit may therefore only take place in tissues where the recombinase is expressed. In this way the reporter may only be expressed in specified tissue types where expression of the recombinase results in a functional transcription unit comprised of the inducible promoter linked to the promoter. Site specific recombinase systems know to perform such a function include the bacteriophage P1 cre-lox and the bacterial FLIP systems. The site specific recombinase sequences may therefore be two loxP sites of bacteriophage P1.

The use of site specific recombination systems to generate precisely defined deletions in cultured mammalian cells has been demonstrated. Gu et al. (Cell 73 1155-1164 (1993)) describe how a deletion in the immunoglobulin switch region in mouse ES cells was generated between two copies of the bacteriophage P1 loxP site by transient expression of the Cre site-specific recombinase, leaving a single loxP site. Similarly, yeast FLP recombinase has been used to precisely delete a selectable marker defined by recombinase target sites in mouse erythroleukemia cells (Fiering et al., Proc. Nat'l. Acad. Sci. USA 90 8469-8473 (1993)). The Cre lox system is exemplified below, but other site-specific recombinase systems could be used.

A construct used in the Cre lox system will usually have the following three functional elements:
1. The expression cassette;
2. A negative selectable marker (e.g. Herpes simplex virus thymidine kinase (TK) gene) expressed under the control of a ubiquitously expressed promoter (e.g. phosphoglycerate kinase (Soriano et al., Cell 64 693-702 (1991)); and
3. Two copies of the bacteriophage P1 site specific recombination site loxP (Baubonis et al., Nuc. Acids. Res. 21 2025-2029 (1993)) located at either end of the DNA fragment.

This construct can be eliminated from host cells or cell lines containing it by means of site specific recombination between the two loxP sites mediated by Cre recombinase protein which can be introduced into the cells by lipofection (Baubonis et al., Nuc. Acids Res. 21 2025-2029 (1993)). Cells which have deleted DNA between the two loxP sites are selected for loss of the TK gene (or other negative selectable marker) by growth in medium containing the appropriate drug (ganciclovir in the case of TK).

According to the further aspect of the invention there is provided a host cell transfected with at least one nucleic acid construct according to any one of the previous aspects of the invention. The cell type is preferably of human or non-human mammalian origin but may also be of other animal, plant, yeast or bacterial origin.

It will be appreciated that the host cell may be transfected with a plurality of reporter systems according to the present invention.

According to a yet further aspect of the invention, there is provided a transgenic non-human animal in which the cells of the non-human animal express the protein encoded by the nucleic acid construct according to any one of the previous aspects of the invention. The transgenic animal is preferably a mouse but may be another mammalian species, for example another rodent, e.g. a rat or a guinea pig, or another species such as rabbit, or a canine or feline, or an ungulate species such as ovine, porcine, equine, caprine, bovine, or a non-mammalian animal species, e.g. an avian (such as poultry, e.g. chicken or turkey).

It will be appreciated that the animals of the present invention may be engineered to comprise more than one reporter system according to the present invention.

In embodiments of the invention relating to the preparation of a transfected host cell or a transgenic non-human animal comprising the use of a nucleic acid construct as previously described, the cell or non-human animal may be subjected to further transgenesis, in which the transgenesis is the introduction of an additional gene or genes or protein-encoding nucleic acid sequence or sequences. The transgenesis may be transient or stable transfection of a cell or a cell line, an episomal expression system in a cell or a cell line, or preparation of a transgenic non-human animal by pronuclear microinjection, through recombination events in embryonic stem (ES) cells or by transfection of a cell whose nucleus is to be used as a donor nucleus in a nuclear transfer cloning procedure.

Methods of preparing a transgenic cell or cell line, or a transgenic non human animal, in which the method comprises transient or stable transfection of a cell or a cell line, expression of an episomal expression system in a cell or cell line, or pronuclear microinjection, infection of a cell or cell lines with a viral vector, recombination events in ES cells, or other cell line or by transfection of a cell line which may be differentiated down different developmental pathways and whose nucleus is to be used as the donor for nuclear transfer; wherein expression of an additional nucleic acid sequence or construct is used to screen for transfection or transgenesis in accordance with any of the aspects of the invention. Examples include use of selectable markers conferring resistance to antibiotics added to the growth medium of cells, e.g. neomycin resistance marker conferring resistance to G418. Further examples involve detection using nucleic acid sequences that are of complementary sequence and which will hybridise with, or a component of, the nucleic acid sequence in accordance with the first, second, third, or fourth aspects of the invention. Examples would include Southern blot analysis, northern blot analysis and PCR.

In an alternative embodiment of the present invention the host cell or transgenic animal may also be engineered to comprise two or more constructs so as to allow a choice of readout or differentiable simultaneous readouts.

Preferably, the secreted/excreted reporter product or metabolite is a product that is excreted in a body fluid of the transgenic animal. For example and without limitation in body fluids such as urine, saliva, tears, milk, cerebrospinal fluid and semen so that its presence is readily assayed and quantified in that fluid in advantageously a non-invasive way. Alternatively the gene product may be assayable in serum, whole blood or tissue of the transgenic animal so that a gene activation event is detected after removal of serum, whole blood or tissue either post mortem or as a procedure during investigation in which case the transgenic animal is not sacrificed.

Preferably, the reporter gene encoding a reporter product or protein or molecule of the present invention possesses characteristics which favour urinary excretion of the reporter moiety.

Preferably, the reporter moiety is of relatively low molecular weight, typically in the region of <120 kDa and more preferably <90 kDa and more preferably still <60 kDa Ideally the reporter moiety possesses a hydrophilic globular tertiary structure, has low bio-activity is stable in urine or the body fluid of choice and is clearly distinguishable from native molecules and is readily detectable and quantifiable.

We have found that SEAP is a suitable secreted/excreted reporter gene for the present invention, however it will be appreciated that other reporter moieties satisfying the above criteria will also be of utility in all the aspects of the present invention.

For example, other secreted/excreted molecules included in the present invention are selected from the group comprising; hormonal molecules, such as human chorionic gonadotrophin or FSH; antibodies such as γ and λ light chain (Bence Jones) proteins, in this particular embodiment of the invention a single chain may be excreted then recombined ex vivo with a partner chain whereby the combination is detectable only ex vivo; and enzymatic molecules such as feline urinary carboxylesterase.

Preferably, one construct of the present invention comprises a modified human β choriogonadotrophin (hCG) molecule. It may also further includes a stratifin gene promoter. The modification may take the form of tagging such as with a myc-tag.

According to a yet further aspect of the invention, there is provided the use of a nucleic acid construct in accordance with any one of the previous aspects of the invention for the detection of a gene activation event resulting from a change in altered metabolic status in a cell in vitro or in vivo.

The gene activation event may be the result of induction of toxicological stress, metabolic changes, disease that may or may not be the result of viral, bacterial, fungal or parasitic infection.

According to a yet further aspect of the invention there is provided the use of a nucleic acid construct comprising a nucleic acid sequence encoding a secreted/excreted protein, wherein said protein is heterologous to the cell in which it is expressed, for the detection of a gene activation event resulting from a change in altered metabolic status in a cell in vitro or in vivo.

The gene activation event may be the result of induction of toxicological stress, metabolic changes, disease that may or may not be the result of viral, bacterial, fungal or parasitic infection.

Uses in accordance with the various aspects of the invention also extend to the detection of disease states or characterisation of disease models in a cell, cell line or non human transgenic animal where a change in the gene expression profile within a target cell or tissue type is altered as a consequence of the disease. Diseases in the context of this aspect of the invention which are detectable under the methods disclosed may be defined as infectious disease, cancer, inflammatory disease, cardiovascular disease, metabolic disease, neurological disease and disease with a genetic basis.

An additional use in accordance with this aspect of the invention involves the growth of a transfected cell line in a suitable immunocompromised mouse strain (referred to as a xenograft), for example, the nude mouse, wherein an alteration in the expression of the reporter described in other aspects of the invention may be used as a measure of altered metabolic status of the host as a result of toxicological stress, metabolic changes, disease with a genetic basis or disease that may or may not be the result of viral, bacterial, fungal or parasitic infection. The scope of this use may also be of use in monitoring the effects of exogenous chemicals or drugs on the expression of the reporter construct.

Aspects of the invention extend to methods of detecting a gene activation event in vitro or in vivo.

In an alternative embodiment of the invention, the method comprises assaying a host cell stably transfected with a nucleic acid construct of the invention, or a transgenic non-human animal of the invention, in which the cell or animal is subjected to a gene activation event that is signalled by expression of a peptide tagged secreted/excreted reporter gene.

In an alternative embodiment of the invention, the method comprises assaying a host cell stably transfected with at least one nucleic acid construct comprising a nucleic acid sequence encoding a secreted/excreted protein, wherein said protein is heterologous to the cell in which it is expressed, or a transgenic non-human animal whose cells express such a construct, in which the cell or animal is subjected to a gene activation event that is signalled by expression of a peptide tagged reporter gene.

Accordingly there is provided a method of screening for, or monitoring of toxicologically induced stress in a cell or a cell line or a non-human animal, comprising the use of a cell, cell line or non human animal which has been transfected with or carries a nucleic acid construct as described above.

Toxicological stress may be defined as DNA damage, oxidative stress, hypoxia, post translational chemical modification of cellular proteins, chemical modification of cellular nucleic acids, apoptosis, cell cycle arrest, hyperplasia, immunological changes, effects consequent to changes in hormone levels or chemical modification of hormones, or other factors which could lead to cell damage.

The present invention advantageously is non-invasive since the reporter moiety is ultimately excretable without recourse to autopsy.

Accordingly, there is also provided a method for screening and characterising viral, bacterial, fungal, and parasitic infection comprising the use of a cell, cell line or non human animal which has been transfected with or carries a nucleic acid construct as described above.

Accordingly, there is additionally provided a method for screening for cancer, inflammatory disease, cardiovascular disease, metabolic disease, neurological disease and disease with a genetic basis comprising the use of a cell, cell line or non human animal which has been transfected with or carries a nucleic acid construct as described above.

In these contexts the cell may be transiently transfected, maintaining the nucleic acid construct as described above episomally and temporarily. Alternatively cells are stably transfected whereby the nucleic acid construct is permanently and stably integrated into the transfected cells' chromosomal DNA.

Also in this context transgenic animal is defined as a non human transgenic animal with the nucleic acid construct as defined above preferably integrated into its genomic DNA in all or some of its cells.

Expression of the peptide tagged, preferably epitope tagged secreted/excreted protein can be assayed for by measuring levels of the protein in cell culture medium or purified or partially purified fractions thereof.

Detection and quantification of the secreted/excreted proteins secreted from cultured cells into tissue culture medium or transgenic non-human animal body fluid may be achieved using a number of methods known to those skilled in the art. For example, immunological methods, such as ELISA or competitive ELISA, Western blot analysis or fluorescence polarisation. Release of a labelled substrate e.g. radioactive (CAT) or fluorometric, colormetric. Detection of multiple substrates by for example mass spectrometry or, nuclear magnetic resonance (NMR).

In a further embodiment of the invention there is provided a method of detecting a reporter gene activation event comprising the steps of:
1. Transfecting a cell or microinjecting the pronucleus of a fertilised mouse egg with a nucleic acid sequence encoding a secreted/excreted protein optionally tagged with a peptide or protein as hereinbefore described and optionally using the microinjected egg or transfected mouse ES cell line;
2. Exposing the transfected cell, cell line or transgenic non human animal to a stimulus which may or may not cause a change in metabolic status resulting alteration in gene expression; and.
3. Using a suitable assay to determine the level expression of the secreted/excreted reporter protein which is optionally tagged, for example using detection methods such as ELISA, RIA, Mass spectrometry, NMR, telemetric methods.

In step (1), the detectable secreted/excreted protein may be a heterologous protein to the cell in which the nucleic acid construct is expressed. Such an “untagged” SEAP reporter protein may not therefore need a peptide or protein tag for detection.

Methods and uses in accordance with the present invention offer significant advances in investigating any area in which modified gene expression plays a significant role. Such reporter genes will be of use in cells and transgenic animals to detect activity of a variety of selected other genes. Specific applications include but are not restricted to:
1. Providing a rapid and robust in vivo screening system for assessing the potential toxic effects of chemicals.
2. Provide information on the mechanism of toxicity. Such information could be used to eliminate compounds from a selection process or suggest possible modifications to a compound.
3. Provide information on the effect of combinations of compounds.
4. Allow monitoring of variation in reporter gene expression over time by measuring levels of reporter(s) in urine at different time intervals.
5. Assessment of changes in gene expression associated with pathogenic infection.
6. Assessment of changes in gene expression associated with neurological, cardiovascular and metabolic diseases.
7. Assessment of changes in gene expression associated with cancer.
8. Provide information allowing validation of drug target selection e.g. by matching reporter expression profile to actions of toxins whose mechanism is defined and understood.
9. Use for evaluating compounds as therapeutic strategies aimed at reversing a toxic, metabolic, or degenerative phenotype.
10. Assessment of changes in gene expression resulting from environmental and/or behavioural changes.

The present invention will now be described with reference to the following examples which are present for the purposes of illustration only and should not be construed as being limited with respect to the invention. Reference in the application is also made the following figures wherein:

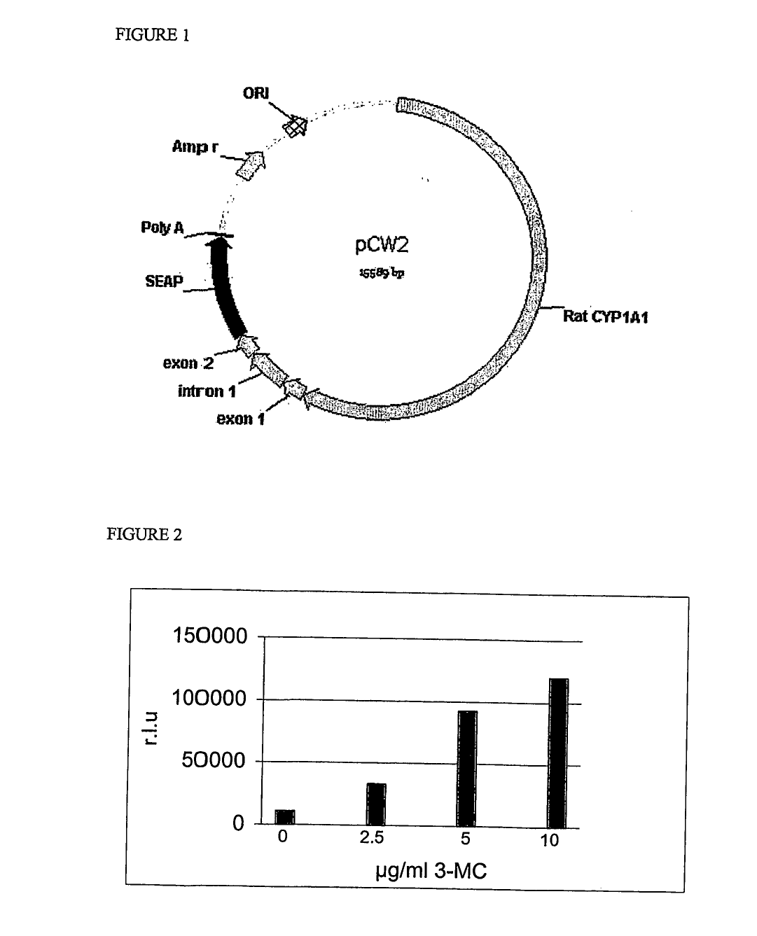

FIG. 1 illustrates the plasmid map for pCW2;

FIG. 2 shows 3MC induction of pCW2 transiently-transfected into Hepa1 cells;

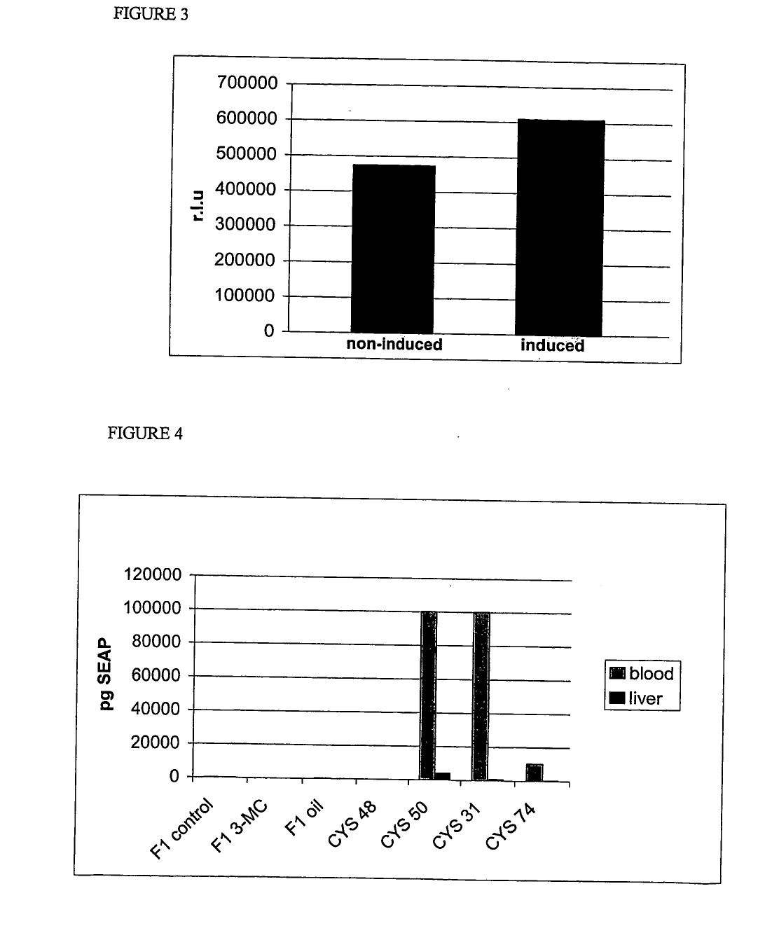

FIG. 3 shows 3MC induction of pCW2 stably-transfected into Hepa1 cells;

FIG. 4 shows 3MC induction of Cyp1a1-SEAP activity detected in urine and blood of founder transgenic animals;

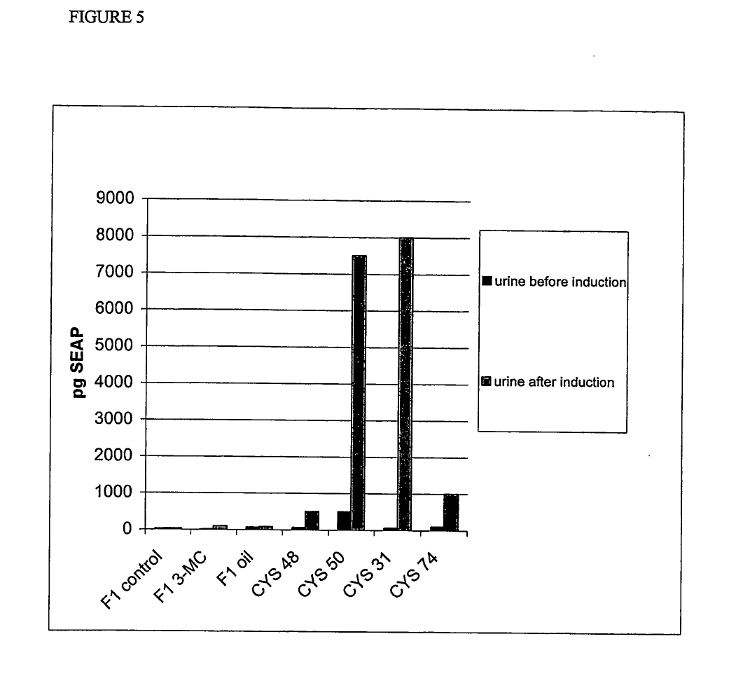

FIG. 5 shows 3MC induced SEAP activity in urine of transgenic animals;

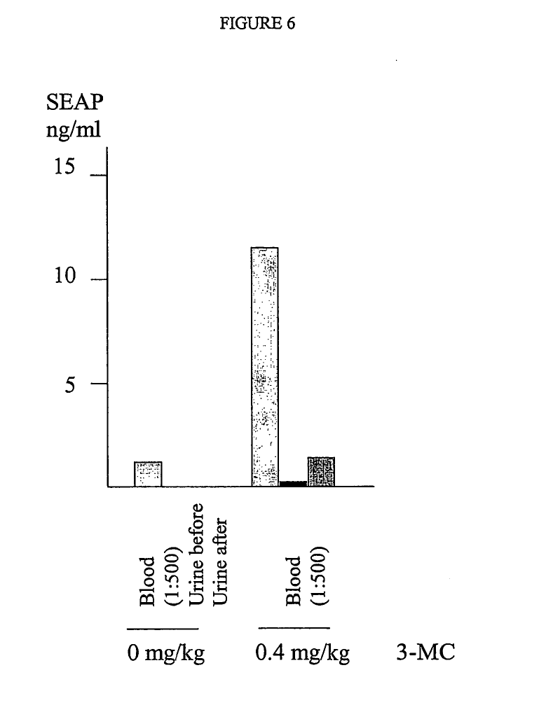

FIG. 6 shows 3MC-induced SEAP activity in the urine of Cyp1A1-SEAP transgenic animals following administration of 0.4 mg/kg 3MC and;

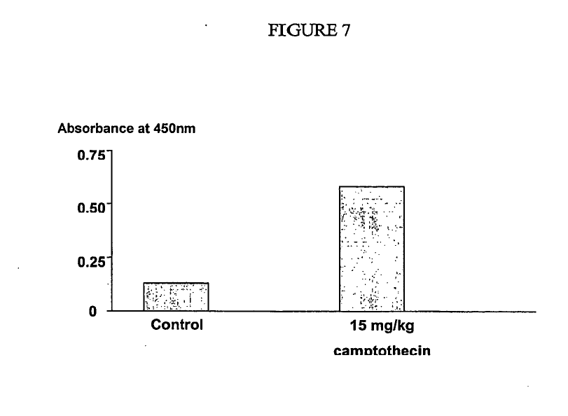

FIG. 7 shows camptothecin-induced increase in hCG(myc) concentrations in the urine of nude mice carrying xenograft tumours of PC3 cells harbouring the SFN-hCG(myc) reporter transgene.

### DETAILED DESCRIPTION OF THE INVENTION

### Example 1

Preparation of pCW2

The sequence coding for 55.5 kD secreted alkaline phosphatase (SEAP), a truncated form of placental alkaline phosphatase enzyme that lacks the GPI anchor, was excised from pSEAP2-Basic (Clonetech) by first converting a BsmI restriction site by removing 3′-overhanging sequences and addition of BglII linker, then excision with BglII. The BglII SEAP fragment was inserted into a linearised pAHIR1 (Campbell et al J. Cell Sci. 109, 2619-2625 (1996)) thereby placing this reporter gene downstream of 8.5 kb of 5′-flanking sequences, the first exon and intron, and the second exon to +2548 of the rat Cyp1A1 promoter. FIG. 1 shows the plasmid map for pCW2.

### Example 2

3MC Induced Cyp1A1-SEAP Vector Expression in Transfected Cells.

Hepa-C1C7 cells (cultured in DMEM supplemented with 10% FCS, 2 mM 1-glutamine, at 5% CO₂) were transfected with pCW2 either transiently or stably (in conjunction with pSVNeo) using the calcium phosphate co-precipitation method. Briefly, 5 μg of plasmid (+1 μg pSVNeo for stable transfections) was mixed with calcium chloride and HEPES buffered saline to form a calcium phosphate-DNA precipitate which was left incubating with the cells for 5 hours. The medium was then replaced with fresh growth medium or with selection medium (growth medium supplemented with 400 μg/ml G418, for stable transfections). Transiently transfected cells were plated into 6 well plates and were incubated with increasing doses of 3MC dissolved in culture medium. For stable transfections, once individual colonies could be identified on the plates the colonies were pooled and incubated with 2 μg/ml 3MC. Forty eight hours after induction with 3MC the medium was assayed for SEAP activity.

SEAP activity was determined using the “SEAP reporter Gene Assay, chemiluminescent” (Roche). Human placental alkaline phosphatase from the kit or from Sigma was used as a positive control. Briefly, samples and standards (0.8 pg-8 μg/ml) were diluted in dilution buffer and heated to 65° C. for 30 minutes. After centrifugation to remove precipitated material the samples were placed on ice and then pipetted into a black 96 well plate (Nunc) together with inactivation buffer for 5 minutes incubation. The activity of the SEAP in each sample was then revealed after a 10 min incubation of the substrate and reading the light emitted with a luminometer (EG&G Berthold 96V microplate luminometer). FIG. 2 shows 3MC induction of pCW2 transiently-transfected into Hepa1 cells and FIG. 3 shows 3MC induction of pCW2 stably-transfected into Hepa1 cells.

### Example 3

Generation of Transgenic Mice with pCW2.

A 10 kb NotI restriction fragment from pCW2 containing the Cyp1A1 promoter, SEAP and polyadenylation sequences was purified by gel electrophoresis, to remove plasmid sequences, and injected into the male pronucleus of fertilized mouse eggs (F1 C57/BL6×CBA) at a concentration of 1.5 ng/μl. Injected eggs that survived culturing to the two-cell stage were transferred to pseudopregnant females (F1) that were allowed to come to term. Genotyping for transgenic status was done by polymerase chain reaction (PCR) on DNA extracted from tail biopsy at 4-6 weeks of age (Whitelaw et al. Transgenic Res 1, 3-13 (1991)) using forward primer 5′-CGCCAAGAACCTCATCATCT-3′ (SEQ ID NO:3) and reverse primer 5′-CGTCAAT GTCCATGTTGGAG-3′ (SEQ ID NO:4) recognising SEAP cDNA sequences.

In one study from 683 eggs injected, 127 pups were born. Twelve (2% of 683) of these pups were identified as transgenic by PCR; 7 females and 5 males.

### Example 4

3MC Induced Cyp1A1-SEAP Vector Expression in Transgenic Mice.

To demonstrate induction for Cyp1A1-SEAP expression in vivo, mice were treated with 3-methylcholanthrene (3MC). Induction followed procedures evaluated previously for ratCYP1A1-LacZ transgenic mice (Campbell et al J. Cell Sci. 109, 2619-2625 (1996)). 3MC was administered to female and male mice (of at least 8 weeks of age) as a suspension in Mazola brand corn oil by i.p. injection. Test animals were either transgenic or non-transgenic age-matched animals with the same genotype were i.p. injected once every 24 hours with either 40 mg/kg or 0.4 mg/kg body weight 3-methylcholanthrene (3MC) in maize oil for three consecutive injections. Control animals were injected with an equal volume of carrier corn oil only. All animals were killed by cervical dislocation 24 hours following the final dose.

Samples of the liver and kidney were removed, washed once in phosphate buffered saline and then homogenised in HB buffer (140 mM NaCl, 50 mM Tris-HCl pH 7.5, 1 mM EDTA, 1% w/v Triton X-100) using a dounce glass homogeniser until a smooth solution was formed. Insoluble proteins were removed by centrifugation at 13000 rpm for 5 minutes and the cleared supernatant was assayed for protein content using the Pierce protein determination kit. For each tissue a final concentration of 0.8 mg/ml was used in the SEAP assay.

Urine and blood samples were centrifuged at 5000 rpm for 5 minutes to remove any solid waste (from the urine) or coagulated cells (from the blood) and were assayed for SEAP activity immediately. Urine was used undiluted in the assay, whereas blood samples were diluted 1:100 and 1:500 with distilled water prior to the SEAP assay. SEAP activity was assayed as described in example 2. FIG. 4 shows induction by 40 mg/kg 3MC of Cyp1a1-SEAP activity detected in urine and blood of 3 out of 4 of founder transgenic mice analysed (CYS48, CYS50, CYS31, CYS74) and FIG. 5 shows 3MC induced SEAP activity in detected in the urine of transgenic mice. FIG. 6 shows the appearance of SEAP in urine of CYS31 transgenic mice following administration of 0.4 mg/kg 3MC.

### Example 5

Generation of a Reporter Gene Encoding an Epitope-Tagged Human Beta-Chorio-Gonadotrophin Protein Under the Control of the Stratifin Promoter.

Epitope-Tagged Human Beta-Chorio-Gonadotrophin Coding Sequence

The following oligonucleotide sequence (designated “myc”):

(SEQ ID NO:5)

CTG CAG GAG CAG AAA CTC ATC TCT GAA GAG GAT CTG
CTG CAG

was inserted at a PstI restriction site in an internal loop of the human beta-chorio-gonadotrophin (hCG) coding sequence between the codons for amino acid residues Val 67 and Gly 68 of the native hCG sequence so that the whole sequence encodes hCG carrying an internal 14 amino acid epitope tag.
Promoter Selection

The promoter of the stratifin (SFN) gene (also known as 14-3-3σ), is a marker of G2/M arrest occurring as a result of DNA damage. The SFN gene has been shown to be transcriptionally upregulated via a p53-dependent mechanism during G2/M arrest in human tumour derived cell lines following γ-irradiation or treatment with adriamycin, also known as doxorubicin (Hermeking H. et al., Molec. Cell 1:3-11, 1997). Expression of SFN appears to be functionally involved in G2/M arrest in that its expression seems to halt progression through the G2/M checkpoint (Hermeking H. et al., Molec. Cell 1:3-11, 1997). In addition, transcriptional activation of the SFN promoter can occur in response to the tumour suppressor protein BRCA1, whose transcriptional activation function is activated by DNA damage (Somasundaran, K., J. Cell Biol, 88:1084-1091, 2003). The facts that SFN induction precedes changes in p53 expression (Aprelikova O. et al, J. Biol Chem, 276:25647-25650, 2001), and that BRCA1 expression is both necessary and sufficient for G2/M arrest and SFN induction in p53-deficient HCC1937 cells (Yarden R. I. et al., Nature Genetics, 30:285-289, 2002), indicate that this pathway of induction is p53-independent. Thus the induction of SFN by DNA damage appears to occur via both p53-dependent and p53-independent pathways (Hermeking H. et al., Molec. Cell 1:3-11, 1997; Aprelikova O. et al, J. Biol Chem, 276:25647-25650, 2001; Yarden R. I. et al., Nature Genetics, 30:285-289, 2002).

Generation of the SFN-hCG Construct

An artificial gene construct was generated in which the hCG(myc) coding sequence was inserted immediately following the ATG start codon of a genomic SFN sequence comprising 10 kb of upstream regulatory promoter DNA and 9 kb downstream sequence. Since the hCG(myc) sequence includes a stop codon, this construct will express hCG(myc) under the control of the SFN promoter. This reporter construct was assembled using recombination cloning utilising the Red/ET homologous recombination system (Genebridges).

The genomic clone of SFN (sfn PROTEIN} was identified using the Human Ensembl site, http://www.ensembl.org/Homo_sapiens, (supported by the Sanger Institute). A human PAC clone RPCI-50o24 was identified to contain the whole coding region and promoter and regulatory regions deemed essential for normal regulation. The PAC clone was further verified by PCR to contain both the 5′ and 3′ UTRs. The SFN oligos used for screening were:

SFN verification oligos to position 48,671-48,690 bp

(SEQ ID NO:6)

SFN_for ATG GTC CTG TGT GTG TCA C

(SEQ ID NO:7)

SEN_rev CAG GGG AAC TTT ATT GAG A

Clones that gave the correct PCR product were then processed as follows. The verified PAC clones were transformed with the plasmid pSC101BADgbaA (Genebridges). This plasmid provides the recombinases essential for the recombination process. The PAC/pSC101BADgbaA clones were further verified for the presence of pSC101BADgbaA by restriction analysis. Only the clones that gave the correct restriction pattern were used.

The generation of hCG(myc):Amp targeting construct was undertaken as follows: The hCG(myc):Amp template had previously been cloned onto the equivalent of the pXEN backbone. This was digested to linearise the template to reduce background. The following oligonucleotides (Bio Spring) were used to generate the targeting molecule:

US-SFNhCG

(SEQ ID NO:8)

TGGTCCCAGGCAGCAGTTAGCCCGCCGCCCGCCTGTGTGTCCCCAGAGCC
ATGGAGATGTTCCAGGGGCTG
LS-SFNamp

(SEQ ID NO:9)

TAGCGTTCGGCCTGCTCTGCCAGCTTGGCCTTCTGGATCAGACTGGCTCT
TTACCAATGCTTAATCAGTGA

The following reaction conditions were used: 39.5 μl dH2O, 5 μl 10× Tuning Buffer (Eppendorf), 2 μl 10 mM dNTPS (Roche), 1 μl US-SFNhCG (100 p mol), 1 μl LS-SFNamp (100 pmol), 0.5 μl Triple Master Taq polymerase (Eppendorf),

PCR Block conditions (MWG): 94° C. for 1 min×1 cycle, 93° C. for 30 seconds, 56° C. for 30 seconds×30 cycles, 72° C. for 2 minutes 30 seconds and 72° C. for 5 minutes×1 cycle

Digestion with Dpn1 was performed on the PCR reaction mixture to preferentially cut the methylated template DNA. The digested PCR reaction was ethanol precipitated and re-suspended in water to give a final DNA concentration of 0.5 ug/ul. The pSC101BADgbaA containing PAC (RPCI-50o24) was cultured as follows; overnight 1 ml LB cultures (supplemented with Kanamycin 70 ug/ml and tetracycline 3 ug/ml) were grown at 30° C. with shaking at 1000 rpm. The next day three 1.4 ml Lb cultures, supplemented as previously, were set up, inoculated with 30 ul of the overnight culture and grown at 30° C. for 2 hours with shaking. After 2 hours two of the cultures were induced with 30 ul of L-arabinose (10%) and all three cultures were shifted to 37° C. with shaking for 1 additional hour (this induces the recombinases and stops the pSC101 BAD plasmid from further replication). The resulting cultures were then treated to make them electrocompetent by three washes in 1 ml if ice cold sterile water. The cells were then electroporated with the PCRed targeting molecule. After electroporation the cells were recovered for 70 minutes with 1 ml of LB broth at 37° C. The recovered cells were then plated out onto LB agar with the selection (Kanamycin 70 ug/ml and Ampicillin 20 ug/ml) and grown overnight at 37° C. The resulting colonies were screened for the correct recombination event (the junctions of hCG and SFN for the 5′ end and Amp and SFN for the 3′ end). On identification of positive clones the pSC101BADgbaA plasmid was re-introduced into the modified PAC and verified as previously described. The next stage was to subclone the modified SFN gene with 10 Kb of upstream sequence and some 9 Kb of downstream sequence onto a pACYC184 backbone. This was again achieved through the use of recombination cloning. The subcloning target construct was generated with PCR using the following oligos:

SFN Subcloning Oligos

SFN subclone forward

(SEQ ID NO:10)

TGCAGTGAGCCGAGATCTCGCCACTGCACTACTCCAGCCTGGGCGACAGA
GCTTACGCCCCGCCCTGCCACTC
SFN subclone reverse

(SEQ ID NO:11)

GGATATGGGAGCCAGCCACATTCATACAGGGCACACATGAACACACACAT
GTCAAACATGAGAATTACAA

PCR conditions were as previously described with the exception of the template used, linearised pACYC184. The PCR product was processed as previously described.

The modified SFN hCG(myc):Amp containing pSC101BADgbaA was made electrocompetent as previously described and electroporated with the SFN subclone intermediate. The resulting transformants were recovered in 1 ml of LB before being plated out onto LB agar plates supplemented with chloramphenicol 15 ug/ml and ampicillin 20 ug/ml. The potential transformants were screened by a number of diagnostic restriction digests and assessed by giving the correct restriction pattern. The clones giving the correct restriction pattern were bulk prepared by growing 400 ml liquid cultures (LB broth supplemented with chloramphenicol 15 ug/ml and ampicillin 20 ug/ml) and maxi prepped using the Qiagen Maxi kit (protocol followed contained within the kit).

### Example 6

Generation of a Cell Line Containing the SFN-hCG(myc)-Amp Reporter Construct

The prostate tumour cell line PC3 is a p53^{-/-} cell line that can be grown as a monolayer in vitro and forms subcutaneous tumours when grown as a xenograft in congenitally athymic nude mice. Importantly, it has the capacity to undergo G2/M arrest following treatment with anticancer drugs (Aranha O. et al., Int. J. Oncol. 22:787-794, 2003).

The SFN-hCG(myc)-Amp reporter construct generated as described under example 5, was transfected into PC3 cells on 6-well tissue culture plates using FuGene reagent (Roche, Lewes, East Sussex). Construct DNA (equivalent to 1 μg/well) was added to FuGene reagent (3 μl/well) and made up to 100 μl/well with serum free medium. The medium in which the cells were growing was aspirated off and replaced with 100 μl of the above mixture per well. After 24 hours the cells were trypsinised and transferred to 10 cm tissue culture dishes (one well of a six well plate per 10 cm dish). The cells were allowed to grow on these dishes for 7 days prior to selection. After this time, G418 (20 ng/ml in cell culture medium) was added and the dishes maintained until colonies became visible (approximately one week). Individual colonies were picked using cloning discs soaked in trypsin and transferred to individual wells of a 24 well plate. The colonies were then expanded to 6 well plates then T25 flasks and grown up until sufficient cells were present for use in in vitro inductions and xenograft experiments.

### Example 7

Detection of Induced SFN-hCG(myc) Transgene Expression from a Xenografted Cell Line In Vivo.

PC3 cells stably transfected with the SFN-hCG(myc)-Amp reporter construct as described under example 6, were allowed to grow as solid subcutaneous tumours in congenitally athymic nude mice. The mice were then treated with anticancer drugs that act by inducing G2/M arrest. The drug chosen for this exemplification was camptothecin (McDonald A. C. and Brown R., Brit. J. Cancer, 78:745-751, 1998). For this experiment, wild-type PC3 cells (which do not express or secrete hCG) and the stable cell line containing the SFN-hCG(myc)-Amp reporter construct were used. Wild-type and engineered PC3 tumour cell lines were cultured in RPMI medium supplemented with 10%-15% heat inactivated foetal calf serum, 2 mM L-glutamine, penicillin (50 IU/ml), streptomycin (50 μg/ml). Culture medium for PC3/SFN cells also contained G418 (200 μg/ml). Cultures were incubated in a humidified incubator at 37° C., 5% CO₂. Cells were harvested, pooled, centrifuged, and re-suspended in cold medium. This was mixed with an equal volume of cold Matrigel, so that the tumour cell injection solution was a 50:50 mixture of tumour cells/medium and Matrigel for each cell line. Wild type or transfected PC3 cells were injected at 2.5×10⁶ per animal. All cell lines were injected in a volume of 100 μl in the right hand flank only.

The study consisted of 4 groups in total, each containing 4 animals. One group of mice was implanted with wild type PC3 cells and the remaining 3 groups with engineered cells. Tumour growth was measured twice-weekly following cell implantation until tumours reached 2-5 mm in diameter. Tumour volume (V) was calculated using the formula: V= 4/3π((d1+d2)/4)³, where d=mean diameter (n=2)

Treatment began 5 weeks after tumour implantation. Wild type PC3-xenografted mice remained untreated; all other mice were administered vehicle only (DMSO/saline) or single i.p. administrations of camptothecin at 15 mg/kg body weight. Urine samples were harvested 24 hours after drug administration. Urine was assayed for hCG(myc) by sandwich ELISA in which hCG(myc) was captured onto the surface of plastic wells coated with a monoclonal antibody against the myc tag sequence (Cancer Research Technologies Ltd.). The hCG(myc) content of individual wells was then assayed incubation with a sheep anti-hCG polyclonal antiserum subsequently labelled with anti-sheep IgG conjugated to horseradish peroxidase (HRP). Quantities of bound HRP were then determined by reaction with tetramethylbenzidine (TMB) and absorbance measurement at 450 nm. No hCG(myc) was detected in the urine of mice carrying xenograft tumours resulting from injection of wild-type PC3 cells. However, hCG(myc) was detected in the urine of mice carrying xenograft tumours resulting from injection of PC3 cells transfected with the SFN-hCG(myc) reporter construct. FIG. 7 shows readout of hCG(myc) concentrations (shown as absorbance at 450 nm) in urine from xenograft mice 24 hours after administration of camptothecin and control urine from mice that had received vehicle solution only. Administration of camptothecin resulted in increased urinary hCG(myc), indicative of transcriptional activation of the SFN-hCG(myc) gene.

1. A nucleic acid construct comprising a nucleic acid sequence further comprising a nucleic acid sequence encoding a reporter protein that is secretable or excretable as a protein or product from a cell where the protein or product is expressed or produced.

2. The nucleic acid construct according to claim 1, wherein the secretable/excretable protein or product is produced by modulated gene transcription.

3. The nucleic acid construct according to claim 1, wherein the secretable/excretable protein or product is produced by increased reporter translation.

4. The nucleic acid construct according to claim 3, wherein the increased reporter translation is a function of increased stability or decreased turnover of mRNA.

5. The nucleic acid construct according to claim 1, wherein the secretable/excretable protein or product is produced by post-translational modulation.

6. The nucleic acid construct according to claim 5, wherein the post-translational modulation is increased reporter stability through removal of polyubiquination or a function of accumulation or excretion of small molecule metabolites.

7. The nucleic acid construct according to claim 37, wherein the peptide tag further comprises an epitope tag or a tag comprising enzymatic activity.

8. The nucleic acid construct according to claim 37 further comprising a promoter element upstream of (i) the nucleic acid sequence encoding the secreted/excreted protein, and/or (ii) a nucleic acid sequence encoding the peptide tag.

9. The nucleic acid construct according to claim 1, wherein the secreted/excreted reporter protein is secreted alkaline phosphatase (SEAP).

10. The nucleic acid construct according to claim 9, wherein the construct further comprises a CypA1 promoter.

11. The nucleic acid construct according to claim 1, wherein the secreted/excreted reporter protein is a modified human β choriogonadotrophin (hCG) molecule.

12. The nucleic acid construct according to claim 11, wherein the construct further comprises a stratifin gene promoter.

13. The nucleic acid construct according to claim 11, wherein the hCG molecule is tagged.

14. The nucleic acid construct according to claim 13, wherein the hCG molecule is myc-tagged.

15. The nucleic acid construct according to claim 1, wherein the secreted/excreted reporter protein/product is selected from the group consisting of hormonal molecules, antibodies and enzymatic molecules.

16. The nucleic acid construct according to claim 15, wherein the hormonal molecule is FSH.

17. The nucleic acid construct according to claim 15, wherein the antibody is γ or light chain (Bence Jones) protein.

18. The nucleic acid construct according to claim 15, wherein the enzymatic molecule is feline urinary carboxylase.

19. A host cell comprising at least one nucleic acid construct according to claim 1.

20. A cell line comprising at least one nucleic acid construct according to claim 1.

21. A transgenic non-human animal wherein the cells of the non-human animal express the protein or product encoded by the nucleic acid sequence of the nucleic acid construct according to claim 1.

22. The transgenic non-human animal according to claim 21, wherein the non-human animal is a mammal.

23. The transgenic non-human mammal according to claim 22, wherein the mammal is a mouse.

24. The transgenic non-human animal according to claim 21, wherein the secreted/excreted reporter protein or product is excreted in a body fluid selected from the group consisting of urine, saliva, tears, milk, cerebrospinal fluid and semen.

25. The transgenic non-human animal according to claim 21, wherein the secreted/excreted reporter protein or product is excreted in urine.

26. The host cell according to claim 19, wherein the secreted/excreted reporter protein or product has a molecular weight of about less than 60-120 kDa.

27. The host cell according to claim 19, wherein the secreted/excreted reporter protein or product comprises a hydrophilic globular tertiary structure, low bio-activity and/or is distinguishable from endogenous molecules.

28-30. (canceled)

31. A method of detecting a gene activation event in a cell in vitro or in vivo, comprising assaying a host cell or transgenic non-human animal each comprising a nucleic acid construct according to claim 1, wherein the cell or animal is subjected to a gene activation event that is signalled by expression of a secreted/excreted reporter protein wherein the protein is optionally tagged with an epitope.

32. A method of screening for, or monitoring of, toxicologically induced stress in a cell or a cell line or a non-human animal, comprising evaluating a cell, cell line or non human animal each comprising the nucleic acid construct according to claim 1.

33. A method for screening and characterizing viral, bacterial, fungal, and parasitic infection or screening for cancer, inflammatory disease, cardiovascular disease, metabolic disease, neurological disease and disease with a genetic basis comprising evaluating a cell, cell line or non human animal each comprising the nucleic acid construct according to of claim 1.

34. The nucleic acid construct according to claim 1, wherein the reporter protein is expressed or produced in vitro or in vivo.

35. The nucleic acid construct according to claim 1, wherein the reporter protein is secretable/excretable from a whole animal.

36. The nucleic acid construct according to claim 35, wherein the whole animal is a transgenic non-human animal.

37. The nucleic acid construct according to claim 1, wherein the construct further comprises a peptide tag.

38. A reporter system comprising at least two nucleic acid constructs, wherein the nucleic acid constructs each comprise:
(i) a nucleic acid sequence encoding a reporter protein; and
(ii) a nucleic acid sequence encoding a peptide tag to the reporter protein,
wherein each reporter protein is distinct from the proteins normally expressed in the host comprising the reporter system.

39. The reporter system according to claim 38, wherein the at least two nucleic acid constructs encode a same reporter protein having a different peptide tag or encode a different protein having a same peptide tag.

40. The reporter system according to claim 38, wherein the reporter protein is selected from the group consisting of secreted alkaline phosphatase (SEAP), a γ or light chain (Bence Jones) protein and feline urinary carboxylase.
